(19) United States
(12) Patent Application Publication
(10) Pub. No.: US 2026/0328724 A1
(43) Pub. Date: Oct. 22, 2026

(54) Salinity Sensor Device Based on a Thin-Film Transistor

(71) Applicant: Shandong Normal University, Jinan (CN)

(72) Inventors: Guoqiang Li, Jinan (CN); Jinghan Sun, Jinan (CN)

(21) Appl. No.: 19/687,015

(22) Filed: May 25, 2026

(51) Int. Cl. G01N 27/414; G01N 27/333; G01N 33/18

(52) U.S. Cl. CPC G01N 27/414; G01N 27/3335; G01N 33/182

(57) ABSTRACT

The present invention provides a salinity sensing apparatus, comprising: a thin-film transistor signal readout unit disposed in a dry area; an ion-sensing gate interface arranged in a wet area; and a water-resistant encapsulation structure configured to physically isolate the dry area from the wet area. The wet area at least includes a reference electrode and an ion-selective membrane. The ion-selective membrane forms a membrane-solution interface with a solution and generates an interfacial potential correlated with salinity. Such interfacial potential is electrically coupled to the gate electrode of the thin-film transistor, so as to modulate the drain-source current of the thin-film transistor or derived characteristic parameters thereof, thereby realizing salinity sensing detection.

### BACKGROUND OF THE INVENTION

Salinity is a critical parameter for characterizing water body properties. Salinity monitoring is widely applied in numerous fields including marine observation, environmental monitoring, aquaculture, marine engineering equipment, in-situ sensing networks, as well as underwater communication and navigation assistance.

Conventional salinity detection methods are mainly based on solution conductivity measurement or electrode-based electrochemical response detection. Such methods are susceptible to adverse factors such as electrode aging, surface fouling deposition, temperature drift and flow velocity disturbance under long-term immersion or complex water environment conditions, which necessitate frequent calibration and routine maintenance. Meanwhile, they also present prominent limitations in terms of miniaturization, low power consumption and array integration applications.

Electrochemical sensors based on field-effect transistors convert variations in interfacial potential into changes in channel carrier concentration, and amplify electrical signals via the transconductance amplification mechanism of transistors, thereby possessing superior advantages including high sensitivity, easy integration and low power consumption. Nevertheless, direct exposure of the transistor channel layer to aqueous solutions tends to cause water penetration, ionic doping contamination, interfacial trap effects, threshold voltage drift and other adverse phenomena, which drastically degrade device operational stability and service life. Such defects become especially severe for solution-processed semiconductors, moisture-sensitive materials and halide perovskite material systems.

Accordingly, there is an urgent need to develop a salinity sensing apparatus and a corresponding salinity sensing method that achieve structural isolation between dry and wet areas, realize efficient gate coupling at the circuit level, and maintain stable output signals under long-term immersion operating conditions.

### SUMMARY OF THE INVENTION

The present invention provides a salinity sensing device capable of converting changes in interfacial potential induced by solution salinity into readable electrical signals of a thin-film transistor, so as to realize amplified and low-noise signal transduction. Meanwhile, physical isolation is established between the sensing region and the transistor readout region to mitigate degradation of semiconductor channel layers and electrode structures caused by water infiltration, ionic contamination, hydrolysis and other adverse factors.

The present invention adopts a water-resistant encapsulation structure which allows the ion sensing interface to be in direct contact with a test solution while preventing the solution from invading and contacting the thin-film transistor channel region. A stable salinity-dependent gate potential is established through an ion-selective membrane and a reference electrode, thereby improving the stability, repeatability and anti-drift capability of the sensor under long-term immersion service conditions.

### BRIEF DESCRIPTION OF THE FIGURES

For a better understanding of the present invention, reference may be made to the following accompanying figures.

FIG. 1 is a structural schematic view of the salinity sensor.

FIG. 2 is a cross section of the salinity sensor.

FIG. 3 is a layered schematic diagram of a composite proton membrane with circular flow-guiding textures and fiber networks.

### DETAILED DESCRIPTION OF THE INVENTION

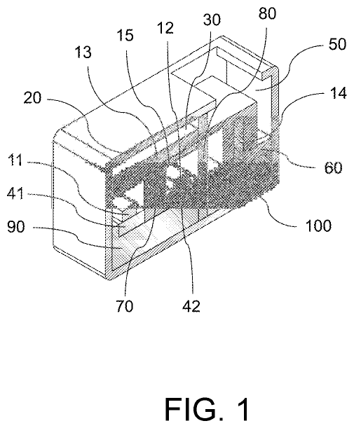

The overall structure of the salinity sensing device of the present invention is illustrated in FIG. 1, which mainly includes a package shell 20, a dry area 30, a wet area 50, and a water-resistant encapsulation structure 80. The dry area 30 is a functional area physically isolated by the water-resistant encapsulation structure 80 and kept away from direct contact with a test liquid. The wet area 50 is a sensing area arranged to come into contact with the test solution to form an ion sensing interface.

The water-resistant encapsulation structure 80 forms a continuous waterproof barrier layer and is provided with a window corresponding to the wet area 50, so that the test solution is only allowed to contact the wet area 50 and is prevented from penetrating into and contacting the dry area 30, thereby achieving a partition isolation arrangement.

Within the wet area 50, the test solution contacts an ion-selective membrane 60 to generate an interfacial potential associated with the ionic activity of the solution at the interface therebetween. Such interfacial potential is coupled to a gate electrode 13 of the thin-film transistor via a reference electrode 14. The reference electrode 14 converts the interfacial potential generated in the wet area into a gate voltage applied across the thin-film transistor.

Variations in solution salinity lead to synchronous changes in ionic activity, interfacial potential and gate voltage, which are ultimately reflected in corresponding shifts in electrical characteristic parameters of the thin-film transistor including drain-source current, threshold voltage and transconductance. Accordingly, a complete sensing signal chain of ion concentration-interfacial potential-transistor electrical signal is established.

Compared with conventional structures where transistors are entirely immersed in test solutions, the present invention realizes physical isolation between dry and wet areas by virtue of the water-resistant encapsulation structure 80. It effectively alleviates performance degradation and structural deterioration of transistor channel materials caused by water permeation, ionic contamination and electrochemical interfacial reactions. The present invention is particularly applicable to thin-film semiconductor material systems with high moisture sensitivity that require stable operation under anhydrous or low-humidity environments, such as halide perovskite channel layers.

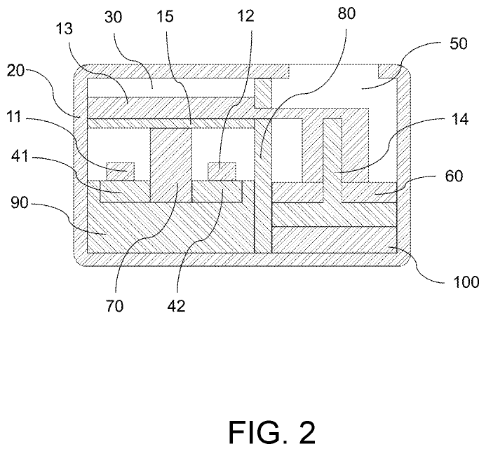

As shown in FIG. 1 and FIG. 2, the device area of the dry area 30 is disposed on the left side of the water-resistant encapsulation structure 80. In service, the physical barrier formed by the water-resistant encapsulation structure 80 keeps the device area of the dry area completely isolated from direct contact with the test solution.

A gate dielectric layer 15 is disposed between the gate electrode 13 and a channel layer 70, which functions to achieve capacitive coupling and suppress gate leakage current. The gate dielectric layer may be formed of silicon dioxide, aluminum oxide, hafnium oxide, silicon nitride, or a multilayer stack thereof. In the present embodiment, the channel layer is formed of an inorganic halide perovskite thin film. The channel layer may also be made of other thin-film semiconductor materials capable of ensuring stable operation of the thin-film transistor.

The channel layer 70 has field-effect electrical modulation characteristics. In the present embodiment, the channel layer 70 is an inorganic halide perovskite thin film. Compared with perovskite materials doped with organic cations, such materials have better structural stability in hot and humid environments. In other embodiments, the channel layer may also be prepared from other component perovskite materials or solution-processable semiconductor materials, as long as the material can ensure the stable operation of the thin-film transistor and can realize integrated assembly with the water-resistant encapsulation structure 80. The thickness of the channel layer 70 ranges from 10 nm to 500 nm, and practical process commonly used thickness specifications such as 40 nm and 150 nm are preferably adopted.

As shown in FIG. 1 and FIG. 2, a source electrode 11 and a drain electrode 12 adopt an interdigital electrode structure. The source electrode 11 is electrically connected to a source highly doped silicon layer 41, the drain electrode 12 is electrically connected to a drain highly doped silicon layer 42, and a lowly doped silicon layer 90 is located below them. The electrode metal material may be Au, Ti, Cr, Pt, etc., and the contact resistance can be reduced through surface treatment. The length of the source electrode 11 and the drain electrode 12 ranges from 1 mm to 1000 mm, and the width ranges from 0.5 mm to 50 mm.

The wet area 50 is the test solution area, which together with the reference electrode 14 and the ion-selective membrane 60 forms an ion sensing gate interface for collecting and transmitting the membrane interface potential. The reference electrode 14 is electrically connected to the gate electrode 13, thereby applying the wet area interface potential to the channel layer 70 located in the dry area 30 through electrical coupling. The material of the reference electrode 14 can be stable conductors such as Pt or Au. Below the reference electrode 14 is a substrate 100.

The ion-selective membrane 60 covers the surface of the reference electrode 14 and is in direct contact with the solution. Taking a cation exchange membrane as an example, the membrane contains fixed anionic groups, which endow the membrane with preferential exchange and transport properties for cations. When the ion-selective membrane 60 comes into contact with the solution, due to the difference in ion activity between the membrane phase and the solution phase, a Donnan equilibrium can be formed at the interface between the ion-selective membrane 60 and the solution, thereby generating an interfacial potential.

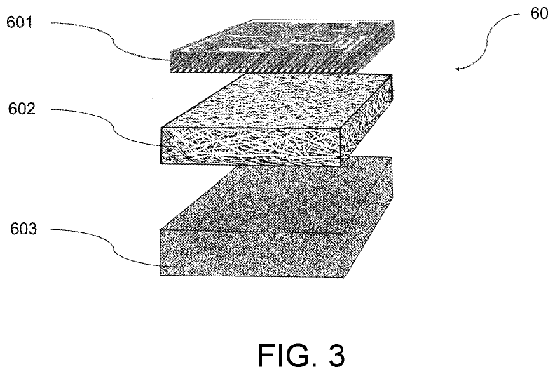

As shown in FIG. 3, the ion-selective membrane 60 is a circular flow-guiding texture-oriented fiber network composite proton membrane, which includes: a circular flow-guiding texture layer 601, a fiber network layer 602, and a transition layer 603. The circular flow-guiding texture layer 601 is located on the outermost side of the ion-selective membrane 60 and is in direct contact with the test solution. It is composed of circular micro-grooves and circular micro-protrusions to form a circular flow-guiding network, which is used to induce the directional spreading and uniform distribution of the test solution on the ion-selective membrane 60, promote the elimination of air bubbles, and suppress output fluctuations and drift caused by local dryness or pollution. The circular flow-guiding texture layer 601 is a hydrophilic anti-fouling polymer layer, a zwitterionic polymer layer, or a cross-linked network layer containing hydrophilic groups, with a thickness ranging from 10 to 500 μm.

The fiber network layer 602 is used to provide ion exchange and serve as a cation transport channel, and realizes the flow guiding and regional uniformization of the solution through the directional arrangement of fibers. In a non-limiting embodiment, the fiber network layer 602 is composed of a plurality of ion exchange fibers containing fixed anionic groups, and the fiber materials are Nafion, sulfonated polyetheretherketone (SPEEK), and sulfonated polyarylether sulfone (SPAES). The fiber diameter ranges from 0.1 to 20 μm, the porosity of the fiber network is 20% to 90%, and the thickness of the main layer is 1 to 200 μm.

The transition layer 603 is used to enhance the binding force between the ion-selective membrane 60 and the reference electrode 14, and suppress interface peeling and liquid seepage paths under long-term immersion conditions. A circular micro-groove structure is formed on the surface of the reference electrode 14, and the bottom of the transition layer 603 penetrates into the micro-grooves and solidifies to form a three-dimensional mechanical interlocking structure; the line width of the micro-grooves may be 1 to 200 μm, the groove depth is 0.1 to 50 μm, and the period is 2 to 1000 μm. The transition layer 603 is a dense barrier layer to prevent the solution from migrating to the dry area along the pore interface.

The water-resistant encapsulation structure 80 is used to isolate the dry area 30 from the wet area 50 and prevent liquid from seeping into the dry area 30. It may include at least one dense inorganic barrier layer and at least one organic sealing layer. The inorganic barrier layer can be deposited with aluminum oxide or hafnium oxide through Atomic Layer Deposition (ALD) technology to achieve low transmittance; the organic sealing layer can be epoxy resin, silicone, or parylene, etc., to achieve mechanical buffering and edge sealing.

1. A salinity sensing device based on a thin-film transistor, comprising:
a dry area, a wet area, a thin-film transistor signal readout unit disposed in said dry area, an ion-sensing gate interface disposed in said wet area, and a water-resistant encapsulation structure physically isolating said dry area and said wet area;
wherein said water-resistant encapsulation structure is provided with a window such that said wet area is directly exposable to a solution under test while preventing said solution from entering and contacting a channel layer and a plurality of electrode structures in said dry area.

2. The salinity sensing device of claim 1, wherein said wet area comprises an ion-selective membrane and a reference electrode;
wherein said ion-sensing gate interface is formed by said reference electrode and said ion-selective membrane, said ion-selective membrane forms a membrane-solution interface with said solution and generates a salinity-dependent interfacial potential, and said interfacial potential is electrically coupled to a gate electrode of said thin-film transistor to modulate a drain-source current of said thin-film transistor, thereby realizing salinity sensing.

3. The salinity sensing device of claim 2, wherein said ion-selective membrane is a composite proton membrane with a circular flow-guiding texture and a fiber network, comprising a circular flow-guiding texture layer, a fiber network layer, and a transition layer.

4. The salinity sensing device of claim 3, wherein said circular flow-guiding texture layer is a hydrophilic anti-fouling polymer layer having a thickness in a range of 10 μm to 500 μm;
said fiber network layer is composed of a plurality of ion-exchange fibers, said fibers having a diameter in a range of 0.1 μm to 20μm and a porosity in a range of 20% to 90%.

5. The salinity sensing device of claim 3, wherein a surface of said reference electrode is formed with a plurality of circular micro-trenches, a bottom portion of said transition layer penetrates into said micro-trenches and is cured to form a three-dimensional mechanical interlocking structure, so as to enhance adhesion between said membrane and said electrode and block liquid penetration paths.

6. The salinity sensing device of claim 1, wherein said thin-film transistor in said dry area comprises a substrate, a gate electrode, a gate dielectric layer, a source electrode, and a drain electrode;
wherein said gate electrode is electrically connected to said reference electrode, and said channel layer is an inorganic halide perovskite thin film having a thickness in a range of 10 nm to 500 nm.

7. The salinity sensing device of claim 6, wherein said gate dielectric layer is selected from the group consisting of silicon dioxide, aluminum oxide, hafnium oxide, and silicon nitride, for capacitive coupling and suppressing gate leakage.

8. The salinity sensing device of claim 1, wherein said water-resistant encapsulation structure comprises a dense inorganic barrier layer and an organic sealing layer;
wherein said inorganic barrier layer is prepared by atomic layer deposition, and said organic sealing layer is selected from the group consisting of epoxy resin, silicone, and parylene.
